(19) United States
(12) Patent Application Publication
(10) Pub. No.: US 2002/0197290 A1
(43) Pub. Date: Dec. 26, 2002

(54) Cosmetic compositions having retarding action on the regrowth of superfluous hair

(76) Inventor: Francesco Di Pierro, Milano (IT)

Correspondence Address: PENNIE & EDMONDS LLP, 1667 K STREET NW, SUITE 1000, WASHINGTON, DC, 20006

(21) Appl. No.: 10/152,805

(22) Filed: May 23, 2002

Related U.S. Application Data
(63) Continuation-in-part of application No. 09/781,301, filed on Feb. 13, 2001, now abandoned.

(30) Foreign Application Priority Data
Mar. 24, 2000 (IT) MI2000A000628

(51) Int. Cl. A61K 6/00; A61K 7/00; A61K 35/78

(52) U.S. Cl. 424/401; 424/725

(57) ABSTRACT

The present invention relates to cosmetic compositions having retarding action on the regrowth of superfluous hair, more particularly to cosmetic compositions containing lipophilic extracts of Serenoa (Serenoa repens) enriched in fatty acids and with a reduced content of sterols.

### CROSS-REFERENCE TO RELATED APPLICATIONS

This application is a continuation-in-part of application Ser. No. 09/781,301, filed Feb. 13, 2001, the contents of which are expressly incorporated herein by reference.

### FIELD OF THE INVENTION

The present invention relates to cosmetic compositions having retarding action on the regrowth of superfluous hair, more particularly to cosmetic compositions containing lipophilic extracts of Serenoa (Serenoa repens) enriched in fatty acids and with a reduced content of sterols.

### BACKGROUND OF THE INVENTION

In humans, the main characteristics concerning the development of hair (number, color, thickness, length, and distribution) involve the consequence of an action mediated by sexual hormones, which in turn, are derived from genetic expression.

In Western Countries, as well as in other socioeconomically advanced Countries, hair of limbs and armpits, pubic hair which can come out of bathing suits, as well as hair which can, although rarely, grow around the mammary areola, is considered unaesthetic in women. The term “superfluous hair” indicates indeed the growth of hair in these areas. As such superfluous hair is of course unwanted, it can be subjected to epilation (removal of the whole hair, roots included, by means of wax or electrolysis) or depilation (removal of the hair starting from the surface of the skin by chemical destruction of keratin, blade or electric shaving, or repeated traumatism of hair). All of these procedures are, however, at least partly traumatic to the skin. These procedures may induce both inflammation in the tissues directly underlying the treated skin and lesions in capillaries and peripheral small vessels. There is therefore the need for such consequences to be reduced as well as for the regrowth of superfluous hair to be retarded as much as possible.

Soler at al. (U.S. Pat. No. 6,113,926) disclose cosmetic formulations containing glycolic or glyceric extracts of Serenoa rich in antiandrogenic sterols, which can be used for the treatment of hirsutism or for controlling and retarding hair loss.

Barr, E. (WO98/33472) discloses pharmaceutical or cosmetic compositions for the prevention or treatment of hirsutism comprising, as an active ingredient, a lipophilic extract of fruits of Serenoa repens plants, which contains a mixtures of fatty acids, long-chained alcohols, sterols and triterpenic alcohols, and the esters thereof.

FR2791255 discloses cosmetic compositions containing vegetal complexes from plants such as Serenoa repens, obtained by means of conventional techniques including extraction with solvents or hypercritical CO₂. The reduction of hair growth is attributed to phytosterols that show 5α-reductase inhibition.

### SUMMARY OF THE INVENTION

One aspect of the present invention relates to a superfluous-hair-growth-retarding cosmetic composition comprising as active ingredient a lipophilic extract enriched in fatty acids and with a reduced sterol content.

Advantageously, the active ingredient(s) are present in a therapeutically or cosmetically effective amount sufficient to provide a retarding action on the growth of superfluous hair. In one preferred embodiment, the amount of the active ingredient(s) present are from 0.1% to about 3% by weight.

The composition may further include anti-inflammatory and/or vasal protective- and/or anti-itching agents, e.g., such as triterpenes from liquorice; saponins from a horse chestnut plant; triterpenes from Terminalia sericea; isobutylamides from Zanthoxylum bungeanum; gingkoflavoneglucosides and terpenes from Ginkgo; polyphenols from grapes seeds; anthocyanosides from bilberries, saponins from butcher's broom; or a mixture thereof. In one embodiment, each anti-inflammatory or vasal protective agent is present in an amount from about 0.1% to about 3% by weight.

The composition may also further include a pharmaceutically or cosmetically acceptable carrier, excipient, or adjunct, preferably including a phospholipid, which can form a complex with the active ingredients, thereby improving absorption or effectiveness.

Advantageously, the composition is in the form of a liquid, a solid, or a semi-solid, e.g., such as a lotion, a milk, a solution, an emulsion, a cream, a paste, a gel, a foam, or a combination thereof.

The composition may also further include one or more conventional depilatory agents, e.g., such as a thiol derivative; thioglycolic or thiolactic acid, or an alkali or alkaline-earth metal salt thereof; ethanolamine thioglycate; aminoethanethiol; mercaptopropionic acid; thiglycic or thioacetic acid; barium sulfate; or a mixture thereof.

The composition may also further include a conventional depilation accelerator, preferably based on a compound containing a urea, a thiourea, or a biguanide, or a combination of such compounds.

Another aspect of the invention relates to a cosmetic treatment method, which includes applying to a region of the body having superfluous hair a cosmetically or therapeutically effective amount of any of the compositions detailed above.

Advantageously, the therapeutically or cosmetically effective amount is sufficient to provide a retarding action on the growth of superfluous hair. In addition, these methods can involve applying from about 0.01 mL to about 0.1 mL of the composition per square centimeter of skin area.

### DETAILED DESCRIPTION OF THE INVENTION

The lipophilic extracts of Serenoa enriched in fatty acids and with a reduced content of sterols according to the invention are prepared as disclosed in EP 250953, which is herein entirely incorporated by reference.

The extractive process is carried out at temperatures ranging from 30° to 50° C., pressures ranging from 100 to 350 bars, and the evaporation at temperatures ranging from 20° to 30° C., at pressures ranging from 50 to 70 bars. The extracts obtained in these conditions contain at least 85% of fatty acids (free or esterified), whereas the content in phytosterols is lower than 0.4% (w/w). The extraction of S. repens fruits with hypercritical CO₂ gives directly an oil that can be used without further purification.

Notwithstanding the extremely low content of phytosterols, the oily extract obtained from the process indicated above showed, during biochemical and pharmacological tests, a significant anti-androgenic, hormone-regulating action.

Object of the present invention is therefore to provide cosmetic composition having retarding action on the regrowth of superfluous hair and containing a lipophilic extract of Serenoa repens with a content of fatty acids higher than 85% (by weight) and a content of sterols lower than 0.4%.

Another aspect of the present invention relates to cosmetic compositions further containing anti-inflammatory and/or vasal protective and/or anti-itching agents, preferably including at least one of: anti-inflammatory triterpenes from liquorice (Glycyrrhiza glabra); anti-edematous and anti-inflammatory saponins from horse chestnut (Aesculus hyppocastanum); triterpenes from Terminalia sericea; analgesic and anti-inflammatory isobutylamides from Zanthoxylum bungeanum (sin Alatum); vasal protective gingkoflavoneglucosides and terpenes from ginkgo (Ginkgo biloba); vasal protective polyphenols from grapes seeds (Vitis vinifera); vasal protective anthocyanosides from bilberries (Vaccinium myrtillus); and vasal protective saponins from butcher's broom (Rescus aculeatus). These preferred compositions of the invention can contain either the above indicated pure components or extracts containing them, preferably in an amount from about 0.1% to about 3% by weight.

In a preferred embodiment, the derivatives listed above are complexed with phospholipids.

The additional ingredients, their phospholipidic complexes and the processes for the preparation thereof, may be found in, inter alia, U.S. Pat. No. 5,547,673; International Publication No. WO 00/02570; and European Patent Nos. EP 0,283,713 and EP 0,275,224, the disclosures of all of which are incorporated herein by express reference hereto.

These derivatives can provide anti-inflammatory, analgesic, vasal tonifying, anti-edematous, and/or tonifying activity, which make them useful in the treatment of inflammatory conditions and vasculopathies, and in dealing with capillary fragility.

“Retarding action” as used herein pertaining to hair regrowth, means an action which induces a delay in hair growth rate, a decrease in the amount of growing hair, or both.

According to the present invention, the cosmetic compositions can be in the form of post-depilatory or post-epilatory compositions, i.e. to be used subsequently depilation or epilation, or they may be included in depilatory formulations.

“Depilatory formulations”, as used herein, means formulations capable of removing hair by the cleavage of keratin disulfide bonds, thereby destroying hair or weakening it to the extent that it can easily be removed (e.g., mechanically by means of a spatula, a sponge or simply by washing with water). Conventional depilatory formulations can further contain as an active ingredient thiol derivatives, such as thioglycolic and thiolactic acids, or an alkali and alkaline-earth metal salts thereof; ethanolamine thioglycate; aminoethanethiol; mercaptopropionic acid; thioglycic and thioacetic acids; barium sulfate; or a mixture thereof.

“Post-depilatory compositions”, as used herein without reference to post-epilatory compositions, should be understood to refer to both post-depilatory and post-epilatory compositions, for the sake of simplicity.

For the preparation of the cosmetic/pharmaceutical compositions of the invention, the active ingredients indicated above are mixed with pharmaceutically/cosmetically acceptable carriers or excipients.

The compositions will be suitably formulated as a liquid, e.g., such as a lotion, a milk, a solution, an emulsion, or the like, or a combination thereof, or as a solid or semi-solid, e.g., such as a cream, a paste, a gel, a foam, or the like, or a combination thereof. For this purpose, the compositions of the invention may also contain pharmaceutically and/or cosmetically acceptable carriers, excipients, or adjuncts conventionally used for the preparation of formulations. These formulation may include those for topical use thus including for example, viscosity agents, such as tragacanth, xanthan gum, gypsum, clay, magnesium oxide, talc, or a combination thereof; chelating agents, such as ethylenediaminotetracetic acid or disodium edetate; emollients, such as cetyl and stearyl alcohols, liquid paraffin, glyceryl stearate, silicon oils, hydrocarbon oils and waxes; wetting agents, such as urea or 1,3-butylene glycol; emulsifiers, such as medium chain triglycerids, acrylic acid polymers, ethoxylated stearyl or cetyl alcohols, polyoxyethylene alkyl ethers, or a mixture thereof; absorption promoters, such as cyclodextrans and liposomes; antioxidants, such as ascorbyl palmitate, tocopherol; preservatives, such as glycerin, propylene glycol; stabilizing agents; or fragrances; or a mixture thereof.

Other compositions according to the invention, such as depilatory compositions, may further contain other conventional additives, for example, depilation accelerators, such as those based on ureas, thioureas and biguanides.

Another aspect of the present invention further relates to a method of cosmetic treatment in order to retard the growth of superfluous hair. The method includes applying a cosmetically effective amount of the compositions of the invention on the body area where unwanted hair grows. The amount of the composition to be applied and the frequency of application will vary depending on the area to treat and on the personal characteristics of the user. Generally, it can range from about 0.01 mL to about 0.1 mL of composition per cm2 of skin area, typically to be applied 1 to about 3 times a day during initial treatment, subsequently reducing the frequency, e.g., to approximately one application on alternate days, depending on the results obtained or desired.

Tests were carried out on healthy female volunteers, of age ranging from about 22 to about 40 years, using: a) post-depilatory compositions according to the invention, for use after superfluous hair had been removed with conventional methods; b) conventional depilatory compositions, containing the compositions of the invention; c) conventional depilatory compositions, containing no compositions of the invention.

In the case of treatments with a) and b), hair regrowth was complete not before 3 weeks, progressively decreasing with the progress of treatment; furthermore, regrown hair appeared weakened. Conversely, in treatment with c), hair regrowth was complete within about 2 to about 3 weeks.

### EXAMPLE

The following examples are only representative of the methods and materials for use cosmetic compositions according to the invention, and are not to be construed as limiting the scope of the invention in any way.

### Example 1

In a 5 L extractor, equipped with heating and with all the accessories for pressure control, 1.2 kg of Serenoa repens fruits, finely ground by a cryocontusion process (cold grinding at −20° C.), were extracted.

The drug was extracted, using subsequently 10 L of continuous recycling CO₂, for 2 hours, at a temperature of 35° and 250 bars. After evaporation of the solvent, the extracted material was recovered and dried at 2 mmHg and 45° C. for 24 hours.

In this way, 0.138 kg of a yellow orange clear oil, with the following characteristics, were obtained:

Thickness: 0.896

Refraction index: 1.46

Substances that cannot be saponified: 2.52%

Saponification index: 230

Content in free fatty acids: 86%.

### Example 2

In a 5 L extractor, equipped with heating and with all the accessories for presurrization and its controls, 1.2 kg of Serenoa Repens fruits finally ground by cryogrinding were extracted as in Example 1.

The drug was on the whole extracted with 10 L of continuous recycling CO₂ for 2 hours at a temperature of 45° C. and 220 bars, keeping a temperature of 25° C. and 50 bars in the condenser.

When the extraction was complete, after a complete evaporation of CO₂ from the condenser, the extracted oil saturated with water was recovered and filtered on 12 g of anhydrous sodium sulphate.

145 g of yellow orange oil were obtained; the oil was dried under vacuum at 2 mm/Hg at 50° for a complete dehydration, and it has the same characteristics as the oil obtained in Example 1.

### Example 3

### Post-Depilatory Gel Formulation

A formulation according to the invention was prepared containing 2% lipophilic extract of serenoa repens, 0.3% ruscogenins, and 0.5% zanthoxylum bungeanum extract. 100 g contain:

| | |
|---|---|
| Serenoa repens* lipophilic extract | 2 g |
| Ruscogenins | 0.3 g |
| 20% zanthoxylum bungeanum** extract solution (in water) | 0.5 g |
| Ethanol | 20 g |
| PEG-6-Caprylic/Capric Glycerides (SOFTIGEN 767, | 15 g |
| from Huls) | |
| Propylene glycol | 10 g |
| Oleth 20 (VOLPO 20, from Croda) | 5 g |
| Dimethicone copolyol | 2.5 g |
| Carbomer | 2 g |
| Triethanolamine | 1 g |
| Zinc ricinoleate (TEGODOR HY77, from Teco) | 0.2 g |
| Menthol | 0.5 g |
| Preservatives | 0.5 g |
| Antioxidants | q.s. |
| Purified water | q.s. to 100 g |

*Prepared according to U.S. Pat. No. 5,547,673.
**Prepared according to International Publication No. WO 00/02570.

### Example 4

### Post-Depilatory Oil-in-Water Emulsion Formulation

A formulation according to the invention war prepared containing 1% glycerrhetinic acid complex with phospholipids, 2% serenoa repens, and 1% ruscogenins. 100 g contain:

| | |
|---|---|
| Glycyrrhetinic acid complex with phospholipids* | 1 g |
| Ruscogenins** | 1 g |
| Serenoa repens lipophilic extract | 2 g |
| Glyceryl stearate (CUTINA GMS, from Henkel) | 7 g |
| Polysorbate 60 (TWEEN 60, from ICI) | 5 g |
| Propylene glycol | 5 g |
| Octyldodecanol (EUTANOL G, from Henkel) | 4 g |
| Wheat germ oil | 3 g |
| PEG-S soy sterols (GENEROL 122 ES, from Henkel) | 2 g |
| Dimethicone | 1 g |
| Carbomer | 0.6 g |
| Tocopherol | 0.2 g |
| Ascorbyl Palmitate | 0.1 g |
| 10% sol. NaOH solution (in water) | 2 g |
| Disodium EDTA | 0.1 g |
| Zinc ricinoleate (TEGODOR CW90, from Tego) | 0.2 g |
| Preservatives | q.s. |
| Fragrance | q.s. |
| Purified water | q.s. to 100 g |

*Prepared according to European Patent No. EP 0283713.
**Prepared according to European Patent No. EP 0250953.

### Example 5

### Post-Depilatory Oil-in-Water Emulsion Formulation

A formulation according to the invention war prepared containing 1.5% glycerrhetinic acid complex with phospholipids, 0.5% Vitis vinifera procyanidolic oligomers complex with phosholipids, and 2% Serona repens lipophilic extract. 100 g contain:

| | |
|---|---|
| Glycyrrhetinic acid complex with phospholipids* | 1.5 g |
| Vitis vinifera procyanidolic oligomers complex with | 0.5 g |
| phospholipids** | |
| Serenoa repens lipophilic extract | 2 g |
| Propylene glycol | 5 g |
| Modified jojoba oil (CETIOL J600, from Henkel) | 5 g |
| Cetyl Alcohol | 5 g |
| Glyceryl stearate (Cutina GMS - Henkel) | 5 g |
| Polysorbate 60 (Tween 60 - ICI) | 3 g |
| Hectorite (and) hydroxyethylcellulose (Betone LT - Rheox) | 0.5 g |
| Dimethicone 350 | 0.5 g |
| Zinc Ricinoleate (TEGODOR CW90, from Tego) | 0.2 g |
| Disodium edetate | 0.1 g |
| Tocopherol | 0.2 g |
| Ascorbyl palmitate | 0.1 g |
| Sodium Metabisulfite | 0.1 g |
| Citric acid | 0.1 g |
| Preservatives | q.s. |
| Fragrance | q.s. |
| Purified water q.s. to 100 g | |

*Prepared according to European Patent No. EP0283713.
**Prepared according to European Patent No. EP0275224.

### Example 6

### Post-Depilatory Alcoholic Lotion Formulation

A formulation according to the invention war prepared containing 0.1% 18/β-glycerrhetinic acid, 1% Gingko bilboa, and 0.5% Serenoa repens. 100 mL contains:

| | | |
|---|---|---|
| 18/β-glycyrrhetinic acid | 0.1 | g |
| Gingko biloba dry extract (containing 20% saponins) | 1 | g |
| Serenoa repens purified extract | 0.5 | g |
| Ciclomethicone | 10 | g |
| PEG-40 Hydrogenated castor oil (CREMOPHOR RH40, from BASF) | 1 | g |
| Zinc Ricinoleate (TEGODOR HY77, from Tego) | 0.2 | g |
| Butylated Hydroxytoluene | 0.05 | g |
| Fragrance | q.s. | |
| Alcohol | q.s. to 100 | mL |

### Example 7

### Post-Depilatory Gel Formulation

A formulation according to the invention war prepared having 2% Serenoa repens lipophilic extract. 100 g contain:

| | |
|---|---|
| Serenoa repens lipophilic extract | 2 g |
| Ethanol | 20 g |
| PEG-6-Caprylic/Capric Glycerides (SOFTIGEN 767, from Huls) | 15 g |
| Propylene glycol | 10 g |
| Oleth 20 (VOLPO 20, from Croda) | 5 g |
| Dimethicone copolyol | 2.5 g |
| Carbomer | 2 g |
| Triethanolamine | 1 g |
| Zinc ricinoleate (TEGODOR HY77, from Teco) | 0.2 g |
| Menthol | 0.5 g |
| Preservatives | q.s. |
| Antioxidants | q.s. |
| Purified water q.s. to 100 g | |

It is to be understood that the invention is not to be limited to the exact configuration as illustrated and described herein. For example, it should be apparent that a variety of materials would be suitable for use in the cosmetic compositions or cosmetic treatment method according to the Detailed Description of the Preferred Embodiments. Accordingly, all expedient modifications readily attainable by one of ordinary skill in the art from disclosure set forth herein, or by routine experimentation therefrom, are deemed to be within the spirit and scope of the invention as defined by the appended claims.

What is claimed is:

1. A cosmetic composition comprising a lipophilic extract of Serenoa repens in an amount sufficient to provide a retarding action on the growth of superfluous hair, wherein said lipophilic extract has a fatty acids content of at least about 85% (w/w) and a phytosterols content of less than about 0.4%.

2. The composition of claim 1, further comprising an anti-inflammatory, vasal protective agent, or anti-itching agent.

3. The composition of claim 2, wherein the anti-inflammatory agent is selected from the group consisting of triterpenes from liquorice (Glycyrrhzia glabra), saponins from horse chestnut plant (Aesculus hyppocastanum), triterpenes from Terminalia sericea, isobutylamides from Zanthoxylum bungeanum (sin Alatum) and mixtures thereof and the vasal protective agent is selected from the group consisting of gingkoflavoneglucosides and terpenes from Ginkgo (Ginko Biloba), polyphenols from grapes seeds (Vitis vinifera), anthocyanosides from bilberries (Vaccinium myrtillus), saponins from butcher's broom (Rescus aculeatus), and mixtures thereof.

4. The compositions of claim 3, wherein each anti-inflammatory agent, vasal protective agent, or anti-itching agent is present in an amount from about 0.1% to about 3% by weight.

5. The composition of claim 2, wherein each anti-inflammatory agent, vasal protective agent, or anti-itching agent is complexed with one or more phospholipids.

6. The composition of claim 1, further comprising a pharmaceutically or cosmetically acceptable carrier or excipient.

7. The composition of claim 2, further comprising a pharmaceutically or cosmetically acceptable carrier or excipient.

8. The composition of claim 1, wherein the lipophilic extract of Serenoa repens is present in an amount of about 0.1% to about 3% by weight.

9. The composition of claim 1, in the form of a liquid, a solid, or a semi-solid.

10. The composition of claim 9, in the form of a lotion, a milk, a solution, an emulsion, a cream, a paste, a gel, a foam, or a combination thereof.

11. The composition of claim 2, in the form of a liquid, a solid, or a semi-solid.

12. The composition of claim 11, in the form of a lotion, a milk, a solution, an emulsion, a cream, a paste, a gel, a foam, or a combination thereof.

13. The composition of claim 1, further comprising a conventional depilatory agents.

14. The composition of claim 2, further comprising a conventional depilatory agents.

15. The composition of claim 1, wherein the conventional depilatory agents comprises a thiol derivative; thioglycolic acid, thiolactic acid, or an alkali or alkaline-earth metal salt thereof; ethanolamine thioglycate; aminoethanethiol; mercaptopropionic acid; thioglycic acid, thioacetic acid; barium sulfate; or a mixture thereof.

16. The composition of claim 2, wherein the conventional depilatory agents comprises a thiol derivative; thioglycolic acid, thiolactic acid, or an alkali or alkaline-earth metal salt thereof; ethanolamine thioglycate; aminoethanethiol; mercaptopropionic acid; thioglycic acid, thioacetic acid; barium sulfate; or a mixture thereof.

17. The composition of claim 1, further comprising a conventional depilation accelerator.

18. The composition of claim 17, wherein the conventional depilation accelerator comprises a urea, a thiourea, a biguanide, or a combination thereof.

19. The composition of claim 2, further comprising a conventional depilation accelerator.

20. The composition of claim 19, wherein the conventional depilation accelerator comprises a urea, a thiourea, a biguanide, or a combination thereof.

21. A method of retarding the growth of superfluous hair on a body comprising applying to a region of the body having superfluous hair the composition of claim 1.

22. A method of retarding the growth of superfluous hair on a body comprising applying to a region of the body having superfluous hair the composition of claim 2.
